(19) United States
(12) Patent Application Publication
(10) Pub. No.: US 2012/0225138 A1
(43) Pub. Date: Sep. 6, 2012

(54) PROCESS UTILIZING A COMBINATION OF AN ACID BLEND, PECTIC ENZYME AND YEAST IN A WINE FERMENTATION CONTAINER TO EXTRACT THE BIOLOGICALLY ACTIVE COMPOUNDS FROM MEDICINAL HERBS

(76) Inventor: Dekui Zhang, Columbus, IN (US)

(21) Appl. No.: 13/039,310

(22) Filed: Mar. 3, 2011

(51) Int. Cl. A61K 36/258; A61P 3/10; A61K 36/00

(52) U.S. Cl. 424/728; 424/725

(57) ABSTRACT

A process of preparing herbal medicines according to the present invention comprises the steps of mixing medicinal herb(s) with boiled water, an acid blend, peptic enzyme, yeast nutrients, corn sugar, yeast in a fermentation container; subjecting the mixture to fermentation for a period of time; centrifuging the mixture to remove remaining yeast and non-dissolved materials; subjecting the fluid to reverse osmosis filtration to remove small molecules. The remaining extract is lyophilized to dry powders, which may be further processed into tablets, capsules, and soft capsules. The present invention also relates to the herbal medicines prepared by said process and uses of these herbal medicines in the treatment of human and/or animal diseases.

### BACKGROUND OF THE INVENTION

1. Field of the Invention

The present invention relates to a process for preparing herbal medicines and, more particularly to a process utilizing a combination of an acid blend, pectic enzyme and yeast in a wine fermentation container to extract the biologically active compounds from medicinal herbs. The present invention also relates to the herbal medicines prepared by said process and uses of these herbal medicines in the treatment and/or prevention of human and/or animal diseases.

2. Description of Related Art

Medicinal herbs have been used in China, India and other countries for thousands of years. Some of the herbs have been subjected to modern scientific research as well as human clinical studies and yielded very promising results. In US, natural healing such as herbal treatment is gaining more popularity during the last decades.

Traditional Chinese medicines are prepared in a traditional pot which is heated at boiling temperature. The extraction efficiency of the active components from the medicinal herbs using this cooking process is low as the process cannot break down most of the large molecules in the forms of polyglycans. In the case of polyphenols, it was estimated that only one third of the quantity existing in the herbs could be extracted out using this traditional method. Furthermore, it was estimated that only about 50% of dietary polyphenols can be absorbed in the small intestine.

There are large varieties of active components in medicinal herbs, with different biological effects. Some of them are polyphenols, flavonoids, tannins, proanthocyanins, coumarins, saponins etc. Most of these compounds exist in large molecules. For example, polyphenols are present in the form of esters, glycosides, or polymers and are not easily absorbed in their natural form (Scalbert et al. J. Nutr. 2000, 130: 2073S-2085S). Glycosylation of these compounds affect their biological properties and absorption. Their bioavailability requires breaking down of these conjugates by acid hydrolysis in the stomach and by microflora in the gut. However, these processes are normally very inefficient due to small exchange area and low density of transporting systems; and the microflora may also degrade the active molecules into simple aromatic acids in the process (Manach et al., American Journal of Clinical Nutrition, 2004, 79: 727-747).

Experiments using surgically treated rats in which absorption was restricted to the stomach showed that absorption at the gastric level is possible for some flavonoids, such as quercetin and daidzein, but not for their glycosides. Most of the glycosides probably resist acid hydrolysis in the stomach and thus arrive intact in the duodenum. Only aglycones and some glucosides can be absorbed in the small intestine, whereas polyphenols linked to a rhamnose moiety must reach the colon and be hydrolyzed by rhamnosidases of the microflora before they can be absorbed.

It is also known that microbial digestion is needed in order to extract the active components from plants. One well known example is grape wine. When the antioxidant activity of grape juices, wines made from the same lot as juices and their major polyphenolic constituents was measured, wines had higher activity than the corresponding grape juices and red wine showed the strongest activity among the grape products tested (Sánchez-Moreno et al., Food Research International, 1999, 32, 6: 407-412). Another example is pomegranate juice. In cultured breast cancer cells, fermented pomegranate juice polyphenols consistently showed about twice the anti-proliferative effect as fresh pomegranate juice polyphenols (Kim et al., Breast Cancer Research and Treatment, 2002, 71, 3: 203-217) Indeed, some of herbal extracts used today are in fact obtained from the microbial digested fraction. The blueberry skin contact fermentation yielded the highest content of anthocyanins and polyphenols and the highest beta-carotene bleaching activity and antiradical activity comparing to juice. Tea short-term fermented with S. bacillaris or S. cinereus enhances the color and content of statin, GABA, and polyphenols (Jeng et al., J. Agric. Food Chem., 2007, 55: 8787-8792).

A process utilizing a multi-enzyme system to hydrolyze raw herbal materials and a herbal medicine prepared by said process in order to increase the extraction of active compounds is disclosed in U.S. Pat. No. 5,595,743 to Wu. The multi-enzyme system comprises cellulases, proteases, amylases, lipases and lysozyme.

U.S. Pat. No. 6,616,951 to Kong discloses a herbal extract comprises an aqueous suspension of a plurality of fermentation products of the herb Angelica sinensis. The suspension is made by suspending crushed Angelica sinensis with active yeast in water to let yeast to ferment the Angelica sinensis.

U.S. Pat. No. 7,645,465 and U.S. Pat. No. 7,754,252 to Kim et al. disclose a method of preparing a pharmaceutical composition comprising fermented ginseng and a lactic fermenting product of ginseng obtained by using said method. The process includes an acid-treatment of ginseng and a bio-converting treatment, such as a lactic acid bacterial fermenting and an intestinal bacterial fermenting process.

Diabetes is a chronic (lifelong) disease marked by high levels of sugar in the blood. Insulin is a hormone produced by the pancreas to control blood sugar. Diabetes can be caused by insufficient secretion of insulin, or resistance to insulin or both. High blood levels of glucose can cause severe problems. Since diabetes is a lifelong disease, concerns of accumulated toxicity come from synthesized medicines motivate diabetic patient to take natural herbal medicines.

WIPO Pub. No. 2009/098702 to Ramchandra discloses a herbal composition of Momordica Charantia along with the other biological additives. The extraction of bilogoical additives along with the main ingredients of the herbal composition is carried out in water at the temperature around 100° C. till it reduces to the particular concentration. WIPO Pub. No. WO 2010/032267 to Krishnan discloses herbal formula for prevention and treatment of diabetes and associated complications. In general, the herbs are pulverized and dispensed as sachets. The content of sachet is to be added to a quantity of water and boiled for a couple minutes for use.

Still a new method of preparing herbal medicines is desired by which the extraction efficiency and bioavailability of the active components in the medicinal herbs can be increased. After conducting intensive scientific research, the inventor came to a conclusion that active components of herbs could be extracted more efficiently when the crushed herbs are put in a wine fermentation container and treated by a blend of acids, pectic enzyme and let the herbal mixture ferment by yeast. The process also increases the bioavailability of the active compounds in the herbal medicines because the process breaks down the large molecules into small active molecules that can be absorbed by gastro-intestinal system. This patent application may cause revolutionary changes in the ways that medicinal herbs are processed. There has been no disclosure or suggestion about a process of preparing herbal medicines for treating or preventing human and/or animal diseases using a combination of acid blend, pectic enzyme, and yeast in a wine fermentation container. The present invention is distinguished from the aforementioned disclosures in that the present invention improves the extraction efficiency and bioavailability of the active ingredients in the herbs by a method involving an acid blend, peptic enzyme, and yeast fermentation.

### SUMMARY OF THE INVENTION

One object of the present invention is to provide a process for preparing herbal medicines which can increase the bioavailability of the medicinal herbs.

Another object of the present invention is to provide a process for preparing herbal medicines which can increase the extraction efficiency of the active components of medicinal herbs.

A furthe object of the present invention is to provide a process for preparing herbal medicines that utilizes microbial fermentation technology.

Still another object of the present invention is to provide new herbal medicines that are prepared by using microbial fermentation technology.

Yet another object of the present invention is to provide new herbal medicines that can treat and/or prevent diabetes.

A process of preparing herbal medicines according to the present invention comprises the steps of weighing and mixing medicinal herb(s) with water, an acid blend, pectic enzyme, yeast nutrients, sugar (preferably corn sugar), yeast; subjecting the mixture to primary fermentation for a length of time; transferring the mixture to a secondary fermentation container and subjecting it to secondary fermentation for a length of time; centrifuging the mixture for a period of time to remove remaining yeast and non-dissolved materials; subjecting the fluid to reverse filtration to remove small molecules. The remaining extract is lyophilized into dry powder, which may be further processed into tablets, capsules, and soft capsules. The primary fermentation may take about one week; if necessary, more sugar (preferably corn sugar) can be added to the primary fermentation container and let it to ferment longer. The secondary fermentation may take about three weeks.

Acid blend is a generic name for any commercially available blend of acids (usually citric, tartaric, and possibly malic acid) sold for the acidification of homemade wines or it can be any mixture of the acids mainly tartaric acid, citric acid and malic acid. During the preparation of herbal medicines according to the present invention, acid blend is used for acidification and acid hydrolysis to break down the large molecules in the herbs. Pectin is a complex carbohydrate chains naturally occurring in fruits that can contribute to the viscosity and haziness of a wine. Pectin can be shortened and solubilized (dissolved) by pectic enzyme. In the case of the currently disclosed method to prepare herbal medicines, pectic enzyme is added to crushed herbs to increase extraction of active ingredients because pectic enzyme can promote structural breakdown in the herbs. It is also used during fermentation to eliminate pectin hazes.

Yeast nutrients are added during the herbal medicine preparation according to the present invention to help invigorate the yeast. The yeast nutrients make the fermentation more active and thorough. The yeast nutrients containing amino acids and proteins as a source of nitrogen and minerals, and vitamins are added to the fermentation container for the purpose of invigorating its fermentation. Yeast constitutes a group of single-celled (unicellular) fungi, a few species of which are commonly used to leaven bread, and/or ferment alcoholic beverages. Yeast converts the corn sugars that are added to the fermentation container and turns it into alcohol and carbon dioxide. Meanwhile, yeast fermentation helps breakdown of large molecules in the medicinal herbs and increase the extraction and bioavailability of the bioactive compounds in the herbal medicines.

A rapid fermentation that is normally known as primary fermentation typically occurs during the first 3 to 7 days of the process, after the yeast is added. On average, 70% to 100% of the fermentation activity occurs during this short period. More corn sugars and time (about a week) are provided to ensure that the fermentation is complete. By the end of the second week, the content is transferred to a secondary fermentation container to continue a steady, slowly fermentation, normally known as secondary fermentation. It normally lasts anywhere from 1 to 4 weeks. An air-lock should be used during this slower period to allow the fermentation to vent without letting oxygen or air-borne microbial contaminants in.

The process for preparing herbal medicines comprises adding about 0-0.2% v/v of acid blend, 0-0.4% v/v of pectic enzyme, 0-0.2% v/v of yeast nutrients, 0-19% w/v of sugar, preferably corn sugar, and about 1-3 bag(s) of yeast to the herbs and water in the fermentation container before the fermentation is started.

The preferred embodiment of the process for preparing herbal medicines comprises adding about 0.1% v/v of acid blend, 0.2% v/v of pectic enzyme, 0.1% v/v of yeast nutrients, 9.5% w/v of sugar, preferably corn sugar, and 1 bag of yeast to the herbs and water in the fermentation container before the fermentation is started.

One of the embodiments disclosed in the present application includes herbal medicines prepared from the medicinal herbs that are known to have clinical efficacy in treating diabetes. A list of medicinal herbs including but not limited to Aloe vera; Ginseng; Gymnema sylvestre; Silybum marianum, Silymarin (Milk thistle); Momordica charantia (Bitter Melon); Cinnamon; Clover; Opuntia streptacantha; Barberry roots (Berberis vulgaris); and Lycium barbarum were selected for diabetic formulary based on their efficacy in human clinical studies.

Momordica Charantia is also known as bitter melon. Folk wisdom has it that bitter melon helps to prevent or counteract diabetes mellitus type 2. Bitter melon contains several bioactive glycosides including charantin, which shows hypoglycemic effect, and terpenoid compounds, which have been found to activate the AMPK, the protein that regulates glucose uptake (a process which is impaired in diabetics) and lectin that has insulin-like activity on the peripheral tissues. Aloe vera leaves has been linked with improved blood glucose levels in diabetics; one of the biological active ingredients, lectin, may be related to said effects. Both American ginseng (Panax quinquefolius) and Asian ginseng (Panax ginseng) roots are known to have therapeutic effectiveness in the treatment of type II diabetes. The major active component that relates to counter diabetes in Gymnema Sylvestrre is gymnemic acids, which have antidiabetic, antisweetener and anti-inflammatory activities. Cinnamon has been reported to have remarkable pharmacological effects in the treatment of Type 2 diabetes mellitus and insulin resistance. The plant material used in the study was mostly from Chinese cinnamon (see Chinese cinnamon's medicinal uses). Recent advancement in phytochemistry has shown that it is a cinnamtannin B1 isolated from C. verum which is of therapeutic effect on Type 2 diabetes. The stem of certain Opuntia have been investigated in the treatment of type II diabetes; some researchers have shown a blood glucose-lowering effect of O. streptacantha.

The % w/v range of the medicinal herbs in the diabetic formulary before fermentation are about 0-8% of Aloe vera, 0-4% of Gingseng, 0-8% of Gymnema sylvestre, 0-8% of Silybum marianum, 0-40% of Momordica charantia, 0-1% cinnamon; 0-1% clover, 0-24% Opuntia streptacantha, 0-4% of Barberry roots, and 0-4% of Lycium barbarum. Water is the solvent used in the formulary.

One of the preferred embodiment for diabetic formulary before fermentation contains the medicinal herbs by w/v of about 4% of Aloe vera, 2% of Gingseng, 4% of Gymnema sylvestre, 4% of Silybum marianum, 20% of Momordica charantia, 0.5% cinnamon; 0.5% clover, 12% Opuntia streptacantha, 2% of Barberry roots, and 2% of Lycium barbarum. Water is the solvent used in the formulary.

The process for preparing herbal medicines of the present invention has no selectivity to raw materials, which adapts to any medicinal herbs. The process of the present invention can apply to any herbal medicine formula in treating or preventing a variety of human and/or animal diseases.

In general, the quantity of herbs, boiled water, acid blend, pectic enzyme, yeast nutrients, corn sugar, and yeast can be scaled up or scaled down. The capacity of fermentation container can be adjusted accordingly. The duration for fermentation can be adjusted according to the nature of the herbs.

The present invention also relates to the herbal medicines prepared by the aforementioned method and uses of these herbal medicines in the treatment and/or prevention of human and/or animal diseases.

The more important features of the invention have thus been outlined in order that the more detailed description that follows may be better understood and in order that the present contribution to the art may better be appreciated. Additional features of the invention will be described hereinafter and will form the subject matter of the claims that follow.

Before explaining at least one embodiment of the invention in detail, it is to be understood that the invention is not limited in its application to the details of construction and the arrangements of the components set forth in the following description or illustrated in the drawings. The invention is capable of other embodiments and of being practiced and carried out in various ways. Also it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

The foregoing has outlined, rather broadly, the preferred feature of the present invention so that those skilled in the art may better understand the detailed description of the invention that follows. Additional features of the invention will be described hereinafter that form the subject of the claims of the invention. Those skilled in the art should appreciate that they can readily use the disclosed conception and specific embodiment as a basis for designing or modifying other structures for carrying out the same purposes of the present invention and that such other structures do not depart from the spirit and scope of the invention in its broadest form.

### DESCRIPTION OF THE PREFERRED EMBODIMENT

### EXAMPLE 1

### Preparation of Herbal Medicines for Treating Diabetes

The following medicinal herbs are pre-treated before weighing. The pre-treatment may include but not limited to collection, cleaning, drying, chopping, grinding, baking, pulverization, etc. and are not discussed in the present application. About 0-2 Kgs of Aloe vera, 0-1 Kg of Ginseng, 0-2 Kgs of Gymnema sylvestre, 0-2 Kgs of Silybum marianum, 0-10 Kgs of Momordica charantia, 0-0.2 Kg of cinnamon, 0-0.2 Kg of clover, 0-7 Kgs of Opuntia streptacantha, 0-1 Kg of barberry roots, and 0-1 Kg of Lycium barbarum are weighed and added to a primary fermentation container. Boiled water is added to the fermentation container to bring the total volume including said herbs to about 20-30 L. About 20-40 mL of acid blend, 30-60 mL of peptic enzyme, 20-40 mL of yeast nutrients, 0-5 Kgs of corn sugar, 1 bag of yeast are added to said fermentation container. The mixture is then allowed to ferment for about one week. Another portion of about 0-5 Kgs of corn sugar is added to the mixture, which is then allowed to ferment for about one more week. By the end of the week, the mixture is transferred to a secondary fermentation container and continues to ferment for about 3 more weeks. The mixture is centrifuged for about 10 minutes to remove the remaining yeast and non-dissolved materials. The liquid or supernatant is then subjected to the reverse osmosis filtration to remove small molecules such as sugar, alcohol, and thus, the volume of the extract is reduced. The resultant extract is lyophilized into dry powders, which may be further processed into tablets or capsules.

### EXAMPLE 2

### Preparation of Herbal Medicines for Treating Diabetes

The preferred diabetic formulary and process comprises about 1.0 Kg of Aloe vera, 0.5 Kg of Ginseng, 1.0 Kg of Gymnema sylvestre, 1.0 Kg of Silybum marianum, 5 Kgs of Momordica charantia, 0.1 Kg of cinnamon, 0.1 Kg of clover, 3.5 Kg of Opuntia streptacantha, 0.5 Kg of barberry roots, and 0.5 Kg of Lycium barbarum are weighed and added to a fermentation container. Boiled water is added to a primary fermentation container to bring the total volume including said herbs to about 26 L. About 30 mL of acid blend, 45 mL of peptic enzyme, 30 mL of yeast nutrients, 2.5 Kgs of corn sugar, 1 bag of yeast are added to said fermentation container. The mixture is then allowed to ferment for at least one week. Another portion of about 2.5 Kgs of corn sugar is added to the mixture, which is then allowed to ferment for about one more week. By the end of the week, the mixture is transferred to a secondary fermentation container and continues to ferment for about 3 more weeks. The mixture is centrifuged for about 10 minutes to remove the remaining yeast and non-dissolved materials. The liquid or supernatant is then subjected to the reverse osmosis filtration to remove small molecules such as sugar, alcohol, and thus, the volume of the extract is reduced. The resultant extract is lyophilized into dry powders, which may be further processed into tablets or capsules.

The process for preparing herbal medicines of the present invention has no selectivity to raw materials, which adapts to any medicinal herbs. The process of the present invention can apply to any herbal medicine formula in treating or preventing a variety of human and/or animal diseases.

In general, the quantity of herbs, boiled water, acid blend, pectic enzyme, yeast nutrients, corn sugar, and yeast can be scaled up and down. The capacity of fermentation container should be adjusted accordingly. The length for fermentation can be adjusted according to the nature of the herbs.

The present invention also relates to the herbal medicines prepared by the aforementioned method and uses of these herbal medicines in the treatment and/or prevention of human and/or animal diseases.

While there have been shown and described and pointed out the fundamental novel features of the invention as applied to the preferred embodiments, it will be understood that the foregoing is considered as illustrative only of the principles of the invention and not intended to be exhaustive or to limit the invention to the precise forms disclosed. Obvious modifications or variations are possible in light of the above teachings. The embodiments discussed were chosen and described to provide the best illustration of the principles of the invention and its practical application to enable one of ordinary skill in the art to utilize the invention in various embodiments and with various modifications as are suited to the particular use contemplated All such modifications and variations are within the scope of the invention as determined by the appended claims when interpreted in accordance with the breadth to which they are entitled.

1. A process of preparing herbal medicines according to the present invention comprising the steps of weighing and blending medicinal herb(s) with water, acid blend, pectic enzyme, yeast nutrients, sugar, yeast in a primary fermentation container to make a mixture; subjecting the mixture to primary fermentation for a length of time; transferring the mixture to a secondary fermentation container and subjecting it to secondary fermentation for a length of time; centrifuging the mixture which contains a fluid and yeast and non-dissolved materials for a period of time to remove remaining yeast and non-dissolved materials; subjecting the fluid to reverse osmosis filtration to remove small molecules; and the remaining fluid is lyophilized into dry powder.

2. The process of claim 1, wherein about 0-0.2% v/v of acid blend, 0-0.4% v/v of pectic enzyme, 0-0.2% v/v of yeast nutrients, 0-19% w/v of sugar, and about 1-2 bag of yeast are added to the herbs and water in the fermentation container before the fermentation is started.

3. The process of claim 1, wherein the preferred quantity are about 0.1% v/v of acid blend, 0.2% v/v of pectic enzyme, 0.1% v/v of yeast nutrients, 9.5% w/v of sugar, and 1 bag of yeast to the herbs and water in the fermentation container before the fermentation is started.

4. The process of claim 1, wherein the primary fermentation takes about one week.

5. The process of claim 1, wherein the secondary fermentation takes about three weeks.

6. The process of claim 1 further comprising a step of adding sugar to the mixture and allowing it to ferment for about one more week before transferring the mixture to a secondary fermentation container.

7. The process of claim 1, further comprising a step of processing the dry powder into tablets, capsules, and soft capsules.

8. The process of claim 1, wherein the fermentation container may be a wine fermentation container.

9. The process of claim 1, wherein the medicinal herbs before fermentation including about 0-8% of Aloe vera, 0-4% of Gingseng, 0-8% of Gymnema sylvestre, 0-8% of Silybum marianum, 0-40% of Momordica charantia,0-1% cinnamon; 0-1% clover, 0-24% Opuntia streptacantha, 0-4% of Barberry roots, and 0-4% of Lycium barbarum; water is the solvent used in the process.

10. The process of claim 1, wherein the medicinal herbs before fermentation preferably comprising about 4% of Aloe vera, 2% of Gingseng, 4% of Gymnema sylvestre, 4% of Silybum marianum, 20% of Momordica charantia, 0.5% cinnamon; 0.5% clover, 12% Opuntia streptacantha, 2% of Barberry roots, and 2% of Lycium barbarum; water is the solvent used in the process.

11. The process of claim 1 can apply to any herbal medicine formula in treating and/or preventing a variety of human and/or animal diseases.

12. The process of claim 1, wherein the quantity of medicinal herbs, water, acid blend, pectic enzyme, yeast nutrients, sugar, and yeast can be scaled up or scaled down.

13. A herbal medicine prepared according to the process recited in claim 1 for treating and/or preventing human and/or animal disease.
